(19) United States
(12) Patent Application Publication
(10) Pub. No.: US 2005/0043583 A1
(43) Pub. Date: Feb. 24, 2005

(54) Endoscopy apparatus

(76) Inventors: Reinmar Killmann, Forchheim (DE); Martin Kleen, Neunkirchen (DE); Rainer Kuth, Herzogenaurach (DE)

Correspondence Address: SCHIFF HARDIN, LLP;PATENT DEPARTMENT, 6600 SEARS TOWER, CHICAGO, IL, 60606-6473, US

(21) Appl. No.: 10/851,963

(22) Filed: May 21, 2004

(30) Foreign Application Priority Data
May 22, 2003 (DE) 103 23 216.8

(51) Int. Cl. A61B 1/04

(52) U.S. Cl. 600/109; 600/130; 600/160

(57) ABSTRACT

An endoscopy apparatus has an endoscopy capsule for acquiring images of the inside of an organ or vessel of the human or animal body that can be wirelessly transmitted to an external receiver, with cameras respectively at both ends that supply separate images from different acquisition directions. At least one of the cameras is movable in the capsule either from side-to-side relative to a central position, or circularly around a central position.

### BACKGROUND OF THE INVENTION

1. Field of the Invention

The present invention concerns an endoscopy apparatus of the type having an endoscopy capsule for acquiring images of the inside of an organ or vessel of a human or animal body, that can be wirelessly transmitted to an external receiver, and wherein receptive cameras are provided at each end that supply separate images from different acquisition directions.

2. Description of the Prior Art

For endoscopic examination, especially of the intestine or the inner intestine surface, the use of an endoscopy apparatus in the form of an endoscopy capsule is known. The patient swallows the endoscopy capsule, and it is passively moved by peristalsis, acquires images of the inner intestine surface with an integrated camera, and transmits them to the body surface via an integrated transmission device and a receiving device. Known endoscopy capsules have at one end a miniaturized single-frame camera that is associated with a power supply in the form of a battery, via which a light source is also operated in order to illuminate the acquired environment. Furthermore, a transmission device is provided via which the transmission of the acquired images to the external receiver ensues.

Such an endoscopy capsule conventionally has been suited only for the examination of the small intestine, because the camera tumbles or wobbles in the inner organs with larger diameter (stomach and large intestine), and only a small portion of the inner surface is recorded by the camera and can be graphically represented in a diagnostically relevant manner. The diagnostic yield during a small intestine examination is approximately 70%, meaning that during the passage of the endoscopy capsule through the small intestine, only approximately 70% of the intestine surface is acquired in a manner such that can be diagnostically evaluated. A significant portion is not recorded, and as a consequence a significant portion of pathological changes can be overlooked (misdiagnosed).

From U.S. Patent Publication No. 2002/0109774, an endoscopy capsule is known in which two separate cameras are disposed at both ends of the capsule and provide exposures from different directions. Each camera is associated with a defined optical path, meaning the respective camera systems are rigid insofar as the acquisition regions are non-variable.

From Japanese Application 2001112701 (Patent Abstracts of Japan), an endoscopy capsule is known in which an image sensor is likewise used which is associated with an adjustable optic that, for focusing purposes, can be moved along the optical axis, defined by the rigid image sensor.

An endoscopy capsule is known from U.S. Patent Publication No. 2003/0023150 in which one camera is arranged in two separate housings these housings being coupled with one another via a flexible connection.

### SUMMARY OF THE INVENTION

An object of the present invention is to provide an endoscopy apparatus having an endoscopy capsule that allows an improved and more intensive examination, in particular of the small intestine, and also of organs of larger diameter that could previously be examined only insufficiently.

This object is achieved in accordance with the invention by an endoscopy apparatus of the initially described type wherein at least one camera can be tilted to the side around its center position (in particular alternately to both sides) to change the acquisition region, or can implement a circular movement around the center position.

Differing from known endoscopy capsules, which have only one camera with a fixedly predetermined acquisition direction, the capsule in the inventive endoscopy apparatus has two cameras aligned in different directions at both ends; an endoscopy capsule with a bidirectional acquisition possibility is thus achieved. Each camera supplies a separate image, which has a number of advantages. The diagnostic yield can be significantly increased, in particular in small intestine examinations. Given movement of the capsule through the small intestine, the front camera (in the direction of movement) acquires the small intestine or the small intestine surface before the passing motion of the capsule. Lesions hidden in the mucous membrane folds are not recognizable, and thus possibly may not be detected in the first exposure. If the capsule now migrates through the small intestine, the intestine wall experiences a distension, which leads to the mucous membrane folds elastically expanding and the lesions and the like hidden therein clearly showing up better. This is also due to mucous and bile on the inner intestine surface being displaced or thinned by the passage of the capsule. Because this area is acquired a second time by the rearward-directed camera, it is possible to detect possible diseases or problem regions from this second exposure. This means the diagnostic yield can be significantly increased because the doubled image data set is acquired during an intestine examination, and each image data set shows the intestine surface in a different state, namely immediately before and immediately after the passage of the capsule, with the surface changes associated therewith. A significantly better and well-founded diagnosis of the small intestine thus can be made by the doctor.

At least one camera is inventively capable of movement to change the acquisition region, and this motion can ensue either automatically as long as the endoscopy capsule is located inside the body, or can be controlled via an external signal from the outside that can be received by the endoscopy capsule. This inventive embodiment enables the acquisition region of at least one camera to be varied either continuously or arbitrarily. For example, it is possible, in the case of automatic operation, the camera is intermittently tilted laterally (relative to a center position in which it is, for example, normally aligned, preferably reciprocally to one side and the other, in order to continuously pan (and thus considerably expand) the acquisition region. This is advantageous because the capsule, which is moved by peristalsis, migrates relatively slowly. An alternative to lateral reciprocation is to allow the camera to process around its center position, meaning the acquisition region changes with a circular motion. Both possibilities lead to a considerable expansion of the acquisition region, and this provides advantage that the intestine wall is acquired from angles changing dependent on the motion, and possible wall structures can be better detected.

The inventive endoscopy capsule also offers a number of advantages in the examination of organs or vessels that are larger in diameter than the diameter of the endoscopy capsule, that possess an external shape similar to a drug capsule. The camera tumbles in these organs or vessels because it is narrower than the vessel/organ; but as a result of the use of the second camera, a doubled number of images exist that show the vessel/organ from different viewing directions. The chance that the organ or vessel wall is acquired by both cameras thus increases (in comparison with known endoscopy capsules) such that the diagnostic yield is in the clinically usable range. It is thus possible with an endoscopy capsule to also examine such organs/vessels that could previously not be diagnosed.

The cameras that are respectively at the ends of the capsule (essentially oblong-cylindrical in shape) can be aligned such that their central optical axes lie on a common axis. This means that both cameras are normally aligned with one another; but their acquisition directions are opposite one another. As an alternative, it is possible for the respective optical axes to be at an angle (non-zero) relative to one another. One camera, for example, can be aligned flush with the longitudinal axis of the capsule, while the second camera is at an angle of, for example, 20°- 60° with regard to this.

Because two cameras are provided, two separate image data blocks are to be transmitted. For this, a separate transmission device to transmit the image data to the external receiver can be associated with each camera. As an alternative, a common transmission device can be used that transmits the image data from both cameras. This has the advantage that fewer components are necessary and the endoscopy capsule can be dimensioned smaller. The energy requirement is also less, such that no additional energy source (battery, etc.) has to be provided. It is also not absolutely necessary to provide a second light source for the second camera. It can be sufficient to arrange the one light source by design such that the acquisition regions of both the first and second cameras are illuminated. Nevertheless, the possibility naturally exists to provide a second light source that is fed via a common energy source.

In the case of a common transmission device, it is ensured that the different image signals can be identified on the receiver side, so it is clear which signal originates from which camera. For this purpose, in an embodiment, the common transmission device is fashioned for alternating transmission of the image data of both cameras. This means that an intermittent transmission operation ensues, thus a time-controlled data transmission, such that, using the time-control scheme, the receiver can clearly detect those images that are provided from the respective cameras. Alternatively or in addition, the common transmission device can transmit the image data of both cameras with different frequencies; thus, a quasi-frequency encoding or identification of the camera-specific image data ensues.

In a further embodiment the common transmission device associates the image data of at least one camera, or one or both cameras themselves associate an identifying characteristic (identifier) with the image data. Thus the digital image data of at least one camera is associated with a digital characteristic that identifies the transmitted image data block as belonging to this camera, which can likewise by recognized by the receiver, and it can, camera-specifically process the image data. This identifying characteristic preferably is generated by changing the signal of one or more predetermined image pixels of a camera. For example, a number of predetermined image pixel signals (for example 10) can be changed such that the respective pixels would provide a black image point which is detected by suitable processing software at the receiver side, and the identification thus can be made. In addition, the image signals an be influenced such that the color or color temperature (shown pixel-by-pixel) changes; the characteristic is thus realized by a specific color change. It is appropriate for the receiver to reconstruct the original signals on the basis of the changed signals, in particular in the case of a color change, in order to re-obtain the original information.

A further alternative for generation of a characteristic is to provide an identifying characteristic detectable, that is in the image, on the camera or an optically transparent coating associated with it. This can be any arbitrary marking visible in the image, for example a point or a cross or the like.

In a further embodiment the endoscopy capsule can be actively moved within the organ or vessel by an external unit cooperating with it. This unit, for example, can be an external magnetic field generator that generates a magnetic field that interacts with a capsule-disposed magnet element, such that the capsule (which follows the field change) can be actively moved (guided) in the organ by changing the external magnetic field. This inventive alternative is in particular appropriate in connection with a possible change of the acquisition region of a camera. The doctor hereby has the possibility to travel back to a specific region that the endoscopy capsule had passed, and to selectively examine again a specific wall region by suitable alignment of the camera.

It is also appropriate for one camera to have a wide-angle lens, so the acquisition region exhibits a very large angle, while the second camera preferably has a telephoto lens that enables it to acquire an examination region significantly enlarged. This is particularly advantageous in connection with the active mobility of the endoscopy capsule by an external movement controller and the (preferably externally applied) adjustment of the camera acquisition region of the camera having the telephoto lens. This advantage is even more pronounced if the focal width of the camera with the telephoto lens, or even the focal width of both cameras, are variable by an external adjustment signal receivable by the endoscopy capsule.

### DESCRIPTION OF THE DRAWINGS

FIG. 1 is an illustration of a first embodiment of an endoscopy apparatus in accordance with the invention, in a schematic sectional view.

FIG. 2 is an illustration of a second embodiment of an endoscopy apparatus in accordance with the invention, in a schematic sectional view.

### DESCRIPTION OF THE PREFERRED EMBODIMENTS

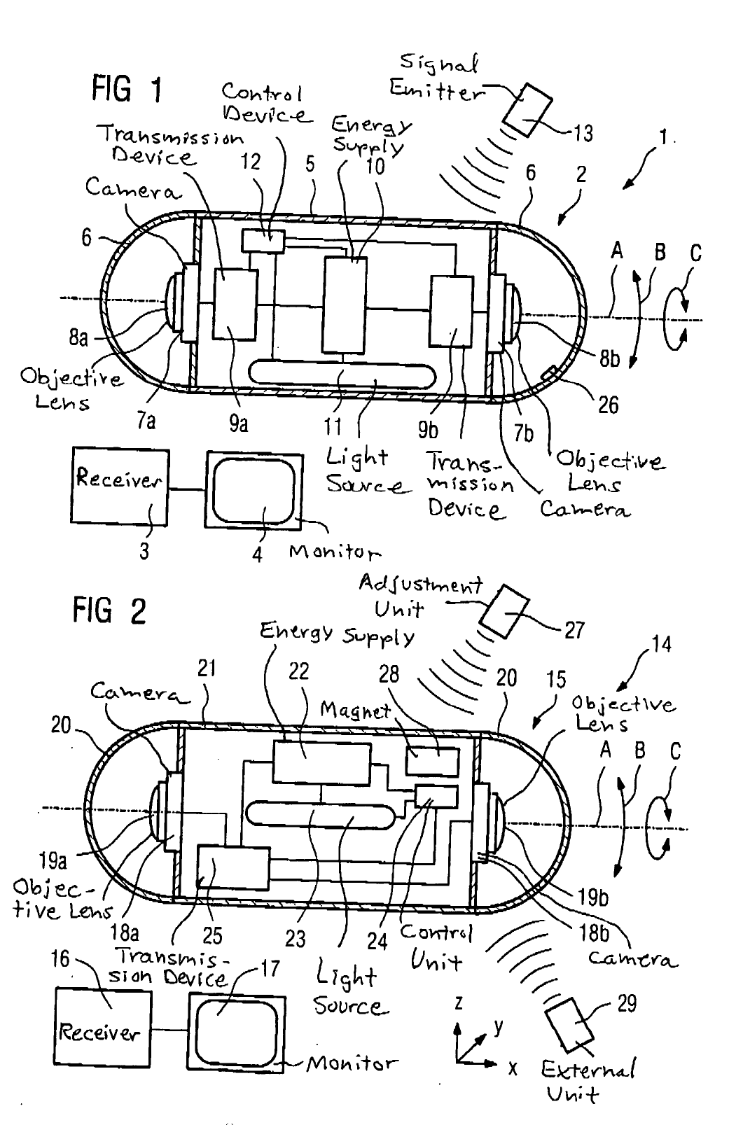

FIG. 1 shows an inventive endoscopy apparatus 1 in a first embodiment having an endoscopy capsule 2 (shown enlarged) to be swallowed by the patient, with a receiver 3 (externally associated with it and preferably to be worn by the patient) to receive image data acquired with the capsule and to process such data, that can be coupled with a monitor 4 serving for image output in order to display the images.

The endoscopy capsule 2 (that is shown in a sectional representation) has a hollow-cylindrical housing 5 that is closed at each end with a transparent, dome-like covering 6. Behind each covering 6 lies a camera 7a, 7b that acquires images of the nearest examination region through the optical transparent covering 6. For this, an objective 8a, 8b is associated with each camera 7a, 7b. The objective 8a, for example, can be a wide-angle lens, while the objective 8b can be a telephoto lens.

Furthermore, a separate transmission device 9a, 9b is associated with each camera 7a, 7b, via which the acquired image data are given through the patient body to the externally positioned receiver 3 that is, for example, attached to the body surface. The receiver 3 is an image data storage in which the image data are acquired and stored during the time in which the endoscopy capsule is located inside the body. The image data are first subsequently read out and displayed on a connectable monitor.

A common energy supply 10 (for example in the form of a battery) via which the cameras 7a, 7b are also supplied is associated with both transmission devices 9a, 9b. A light source 11 (which is preferably a light source 11 flashing with a short period, as a type of stroboscope) is also supplied via the energy supply 10. A control device 12 is provided that controls the overall operation of the endoscopy capsule 2. The operation of the light source 11 is controlled by it, as well as the acquisition operation of the cameras 7a, 7b, which acquire an image precisely when a light flash is emitted by the light source 11. The light source 11 is disposed such that the generated light escapes from both coverings 6 and illuminates the surrounding area near the respective camera.

As FIG. 1 shows, both cameras 7a, 7b are arranged with one another on the axis A. In the simplest case, both cameras 7a, 7b are rigid, thus non-variable with regard to their alignment of the acquisition region. In the inventive embodiment shown in FIG. 1, however, the camera 7b can be pivoted (as is shown by the double arrow B) around the center position in which it is aligned along the axis A, namely to both sides. As an alternative, the camera 76 (as is shown by the double arrow C) can be positioned such that it can be rotated around this center position. The movement operation of the camera 7b can ensue automatically, but it is also possible to induce this movement by an external signal for which, for example, the control device 12 has a suitable receiver module that receives signals emitted from an external signal emission unit and thus induces the motion operation of the camera 7b.

FIG. 2 shows a further inventive endoscopy apparatus 14, likewise having an endoscopy capsule 15 as well as an external receiver 16 with likewise associated monitor 17. The capsule here also has two cameras 18a, 18b with respectively associated objectives 19a, 19b and coverings 20. In addition to the components already described in FIG. 1 and arranged inside the housing 21, namely the power supply 22, the light source 23 and the control device 24, a common transmission device 25 is provided here that transmits the image signals of both cameras 18a, 18b. In order to be able to identify on the receiver side whether the transmitted image data packet now originates from the camera 18a or 18b, or in order to later (in the framework of the image processing) process in common with one another those data blocks that have arrived from the one or the other camera, the transmission operation can ensue time-controlled via the transmission device 25, thus intermittently. This means the transmission of the image data, for example of the camera 18a, first ensues after triggering of a light flash and acquisition of the images, whereupon said image data of the camera 18b are first transmitted time-delayed. It is alternatively possible for the transmission device 25 to transmit the image data on different frequencies, or a camera-specific characteristic or the like is associated with the respective image data. In addition to this, it is naturally also conceivable that—see FIG. 1—a characteristic 26 that is visible in the image of the camera is provided on the optically transparent cover 6, and using which the identification can ensue in the framework of the later image processing. For example, suitable processing software can automatically detect the characteristic in the acquired image data set and corresponding associate the image data. In addition to this, it is naturally also possible for the signal of various image pixels to be selectively influenced at the camera or at the transmission device so as to generate a characteristic.

As FIG. 2 also shows, the camera 18b can be pivoted by an external adjustment unit 27, or to allow it to rotate, insofar as this does not already automatically ensue.

The endoscopy capsule 15 from FIG. 2 also shows a magnet 28 integrated at the capsule, with which an active guidance of the endoscopy capsule 15 is possible inside the organ/vessel. For this, an external magnetic field is generated by an external unit 29 (shown by the small coordinate system x, y, z). This external magnetic field interacts with the integrated magnet 28. If the external magnetic field is changed, the endoscopy capsule 15 follows the magnetic field and can be actively moved in the organ/vessel. This enables it, for example, to be guided back to an already-passed location, and this—in particular in connection with the mobility of the camera 18b—to be more precisely examined. If the objective 19b is a telephoto lens, an examination region can be selectively approached and precisely observed in enlarged display. This is naturally primarily possible when a continuous observation of the supplied images ensues during the time in which the endoscopy capsule is located in the region of interest, such that it can be reacted to quickly.

Although modifications and changes may be suggested by those skilled in the art, it is the intention of the inventors to embody within the patent warranted hereon all changes and modifications as reasonably and properly come within the scope of their contribution to the art.

1. An endoscopy apparatus comprising:
an endoscopy capsule having a size and a shape for allowing said capsule to be swallowed by a subject, said endoscopy capsule having opposite ends;
a first camera and a second camera disposed in said endoscopy capsule respectively at said opposite ends, said opposite ends of said endoscopy capsule being optically transparent for allowing each of said first and second cameras to acquire images, within an associated image acquisition region of an interior of the body of the subject;
at least one of said first and second cameras being mounted for movement in said endoscopy capsule selected from the group of movements consisting of side-to-side movement with respect to a center position and circular movement around a center position, for changing the associated image acquisition region of said at least one of said first and second cameras; and
a transmitter in said endoscopy capsule adapted for wireless communication with a receiver disposed externally of the subject for wirelessly transmitting data representing said images to said external receiver.

2. An endoscopy apparatus as claimed in claim 1 wherein each of said first and second cameras has a central optical axis, and wherein said central optical axes are co-linear.

3. An endoscopy apparatus as claimed in claim 1 wherein each of said first and second cameras has a central optical axis, and wherein said central optical axes form a non-0° and non-180° angle.

4. An endoscopy apparatus as claimed in claim 1 comprising a control unit for controlling movement of said at least one of said first and second cameras, and a receiver in said endoscopy capsule adapted to wirelessly receive control signals from an external signal source for supply to said control unit for controlling the movement of said at least one of said first and second cameras.

5. An endoscopy apparatus as claimed in claim 1 comprising a control unit contained in said endoscopy capsule for automatically controlling the movement of said at least one of said first and second cameras.

6. An endoscopy apparatus as claimed in claim 1 wherein said transmitter comprises a first transmitter unit connected to said first camera for wirelessly transmitting first image data representing an image acquired by said first camera, and a second transmitter unit connected to said second camera for wirelessly transmitting second image data representing an image acquired by said second camera.

7. An endoscopy apparatus as claimed in claim 1 wherein said transmitter comprises a single transmitter unit connected to each of said first and second cameras.

8. An endoscopy apparatus as claimed in claim 7 wherein said single transmitter unit alternatingly wirelessly transmits data representing an image acquired by said first camera and data representing an image acquired by said second camera.

9. An endoscopy apparatus as claimed in claim 7 wirelessly transmits, at a first frequency, data representing an image acquired by said first camera and wirelessly transmits, at a second frequency, data representing an image acquired by said second camera.

10. An endoscopy apparatus as claimed in claim 7 wherein said single transmitter unit associates, and wirelessly transmits, a first identifying characteristic with data representing an image acquired by at least one of said first and second cameras.

11. An endoscopy apparatus as claimed in claim 10 wherein said image acquired by said at least one of said first and second cameras is comprised of a plurality of image pixels, and wherein said single transmission unit changes at least one of said image pixels in the image acquired by said at least one of said first and second cameras as said identifying characteristic.

12. An endoscopy apparatus as claimed in claim 11 comprising said receiver, and wherein said receiver reconstructs said image from said at least one of said first and second cameras by restoring said at least one changed image pixel to an original condition,

13. An endoscopy apparatus as claimed in claim 7 wherein at least one of said first and second cameras associates an Identifying characteristic in the image acquired by said at least one of said first and second cameras.

14. An endoscopy apparatus as claimed in claim 13 wherein the image acquired by said at least one of said first and second cameras is comprised of a plurality of image pixels, and wherein said at least one of said first and second cameras changes at least one of said image pixels as said identifying characteristic.

15. An endoscopy apparatus as claimed in claim 14 comprising said receiver, and wherein said receiver reconstructs said image from said at least one of said first and second cameras by restoring said at least one changed image pixel to an original condition.

16. An endoscopy apparatus as claimed in claim 7 wherein at least one of said first and second cameras has a lens with an optical marking thereon that is contained in the image acquired by said at least one of said first and second cameras as an identifier.

17. An endoscopy apparatus as claimed in claim 7 wherein said endoscopy capsule has a first transparent dome disposed at one end of said endoscopy capsule through which said first camera acquires images, and a second optically transparent dome disposed at an opposite end of said endoscopy capsule through which said second camera acquires images, and wherein at least one of said first and second domes has an optical marking thereon that is included in the image acquired by the respective first or second camera associated therewith, as an identifier.

18. An endoscopy apparatus as claimed in claim 1 further comprising an external unit for generating a guiding field within which said endoscopy capsule interacts for guiding movement of said endoscopy capsule in said subject.

19. An endoscopy apparatus as claimed in claim 1 wherein said first camera has a wide-field lens and wherein said second camera has a telephoto lens.

20. An endoscopy apparatus as claimed in claim 13 wherein said one of said first and second cameras having said telephoto lens has a focal with that is variable using said telephoto lens, and wherein said endoscopy capsule contains a receiver adapted to receive control signals from an external signal source for varying said focal width.
